(19) United States
(12) Patent Application Publication
(10) Pub. No.: US 2016/0317749 A1
(43) Pub. Date: Nov. 3, 2016

(54) ASSEMBLY FOR A DRUG DELIVERY DEVICE AND DRUG DELIVERY DEVICE

(71) Applicant: SANOFI-AVENTIS DEUTSCHLAND GMBH, Frankfurt am Main (DE)

(72) Inventors: Michael Jugl, Frankfurt am Main (DE); Axel Teucher, Frankfurt am Main (DE); Marc Holtwick, Frankfurt am Main (DE)

(21) Appl. No.: 15/105,251

(22) PCT Filed: Dec. 18, 2014
(86) PCT No.: PCT/EP2014/078419

§ 371 Date: Jun. 16, 2016

(30) Foreign Application Priority Data
Dec. 20, 2013 (EP) 13198708.3

(51) Int. Cl. A61M 5/315; A61M 5/20

(52) U.S. Cl. CPC A61M 5/31513; A61M 5/2033; A61M 5/31501; A61M 5/31553; A61M 5/31583; A61M 2005/2086

(57) ABSTRACT

An assembly for a drug delivery device (1) is described comprising a piston rod (12) adapted and arranged to be displaceable in a delivery direction for dispensing a dose of a drug (10) from the device (1), an energy storing member (16) which is adapted and arranged to store energy and to move the piston rod (12) in the delivery direction, and an interaction member (7) adapted and arranged to mechanically cooperate with the piston rod (12), wherein the interaction member (7) is displaceable between a first position and a second position with respect to the piston rod (12), wherein the interaction member (7) is configured to be moved from the first position towards the second position to increase a friction onto the piston rod (12) and, wherein the interaction member (7) is configured to be moved from the second position back into the first position to decrease the friction onto the piston rod (12). Furthermore, a drug delivery device (1) comprising the assembly is described.

The present disclosure relates to an assembly for a drug delivery device. The present disclosure further relates to a drug delivery device. In particular, the disclosure relates to pen-type drug delivery devices.

Pen-type drug delivery devices are used for injections by persons without formal medical training. This is increasingly common for self-treatment among patients having diabetes or the like. By means of a drive mechanism, a bung in a cartridge is displaced such that a drug accommodated in the cartridge is dispensed through a needle.

Prior to injection, the required dose of the drug is set by means of a dose setting mechanism. Common designs of dose setting mechanisms comprise a number of tubular or sleeve-like elements such as a dose setting sleeve, a dose indicating sleeve, a drive sleeve and/or a ratchet sleeve. Such sleeves are often accommodated within and connected to each other. Some devices comprise a power assist, in particular an energy storing member, wherein energy may be stored in the energy storing member during the setting of a dose. This energy may be released during dose delivery.

A power assisted drug delivery device is described in document US 2008/0306446 A1, for example.

Power assisted drug delivery devices are beneficial, in particular when the injection of larger volumes and/or highly viscous fluids is intended. The energy storing member must have a force profile sufficient to enable delivery of the entire dose. This has the impact that at least small doses are dispensed with high force and, therefore, in a fast manner. Fast injections may lead to user discomfort, as the injected tissue is stressed and the user may even feel pain.

It is an object of the present disclosure to provide a drug delivery device having improved properties, e.g. increased user comfort.

This object may be achieved by the subject matter of the independent claims. Advantageous embodiments and refinements are subject matter of the dependent claims.

One aspect relates to an assembly for a drug delivery device. The assembly may comprise a drive mechanism of the device. A drive mechanism may be a mechanism adapted for setting and/or dispensing a dose of drug from the device. The assembly may comprise a piston rod. The piston rod may be adapted and arranged to be displaceable in a delivery direction for dispensing a dose of a drug from the device. The term “drug”, as used herein, preferably means a pharmaceutical formulation containing at least one pharmaceutically active compound,

wherein in one embodiment the pharmaceutically active compound has a molecular weight up to 1500 Da and/or is a peptide, a proteine, a polysaccharide, a vaccine, a DNA, a RNA, an enzyme, an antibody or a fragment thereof, a hormone or an oligonucleotide, or a mixture of the above-mentioned pharmaceutically active compound,

wherein in a further embodiment the pharmaceutically active compound is useful for the treatment and/or prophylaxis of diabetes mellitus or complications associated with diabetes mellitus such as diabetic retinopathy, thromboembolism disorders such as deep vein or pulmonary thromboembolism, acute coronary syndrome (ACS), angina, myocardial infarction, cancer, macular degeneration, inflammation, hay fever, atherosclerosis and/or rheumatoid arthritis,

wherein in a further embodiment the pharmaceutically active compound comprises at least one peptide for the treatment and/or prophylaxis of diabetes mellitus or complications associated with diabetes mellitus such as diabetic retinopathy,

wherein in a further embodiment the pharmaceutically active compound comprises at least one human insulin or a human insulin analogue or derivative, glucagon-like peptide (GLP-1) or an analogue or derivative thereof, or exendin-3 or exendin-4 or an analogue or derivative of exendin-3 or exendin-4.

Insulin analogues are for example Gly(A21), Arg(B31), Arg(B32) human insulin; Lys(B3), Glu(B29) human insulin; Lys(B28), Pro(B29) human insulin; Asp(B28) human insulin; human insulin, wherein proline in position B28 is replaced by Asp, Lys, Leu, Val or Ala and wherein in position B29 Lys may be replaced by Pro; Ala(B26) human insulin; Des(B28-B30) human insulin; Des(B27) human insulin and Des(B30) human insulin.

Insulin derivates are for example B29-N-myristoyl-des(B30) human insulin; B29-N-palmitoyl-des(B30) human insulin; B29-N-myristoyl human insulin; B29-N-palmitoyl human insulin; B28-N-myristoyl LysB28ProB29 human insulin; B28-N-palmitoyl-LysB28ProB29 human insulin; B30-N-myristoyl-ThrB29LysB30 human insulin; B30-N-palmitoyl-ThrB29LysB30 human insulin; B29-N-(N-palmitoyl-Y-glutamyl)-des(B30) human insulin; B29-N-(N-lithocholyl-Y-glutamyl)-des(B30) human insulin; B29-N-(ω-carboxyheptadecanoyl)-des(B30) human insulin and B29-N-(ω-carboxyhepta-decanoyl) human insulin.

Exendin-4 for example means Exendin-4(1-39), a peptide of the sequence H His-Gly-Glu-Gly-Thr-Phe-Thr-Ser-Asp-Leu-Ser-Lys-Gln-Met-Glu-Glu-Glu-Ala-Val-Arg-Leu-Phe-Ile-Glu-Trp-Leu-Lys-Asn-Gly-Gly- Pro-Ser-Ser-Gly-Ala-Pro-Pro-Pro-Ser-NH2.

Exendin-4 derivatives are for example selected from the following list of compounds:

H-(Lys)4-des Pro36, des Pro37 Exendin-4(1-39)-NH2,

H-(Lys)5-des Pro36, des Pro37 Exendin-4(1-39)-NH2,

des Pro36 Exendin-4(1-39),

des Pro36 [Asp28] Exendin-4(1-39),

des Pro36 [IsoAsp28] Exendin-4(1-39),

des Pro36 [Met(O)14, Asp28] Exendin-4(1-39),

des Pro36 [Met(O)14, IsoAsp28] Exendin-4(1-39),

des Pro36 [Trp(O2)25, Asp28] Exendin-4(1-39),

des Pro36 [Trp(O2)25, IsoAsp28] Exendin-4(1-39),

des Pro36 [Met(O)14 Trp(O2)25, Asp28] Exendin-4(1-39),

des Pro36 [Met(O)14 Trp(O2)25, IsoAsp28] Exendin-4(1-39); or

des Pro36 [Asp28] Exendin-4(1-39),

des Pro36 [IsoAsp28] Exendin-4(1-39),

des Pro36 [Met(O)14, Asp28] Exendin-4(1-39),

des Pro36 [Met(O)14, IsoAsp28] Exendin-4(1-39),

des Pro36 [Trp(O2)25, Asp28] Exendin-4(1-39),

des Pro36 [Trp(O2)25, IsoAsp28] Exendin-4(1-39),

des Pro36 [Met(O)14 Trp(O2)25, Asp28] Exendin-4(1-39),

des Pro36 [Met(O)14 Trp(O2)25, IsoAsp28] Exendin-4(1-39),

wherein the group -Lys6-NH2 may be bound to the C-terminus of the Exendin-4 derivative;

or an Exendin-4 derivative of the sequence

des Pro36 Exendin-4(1-39)-Lys6-NH2 (AVE0010),

H-(Lys)6-des Pro36 [Asp28] Exendin-4(1-39)-Lys6-NH2,

des Asp28 Pro36, Pro37, Pro38Exendin-4(1-39)-NH2,

H-(Lys)6-des Pro36, Pro38 [Asp28] Exendin-4(1-39)-NH2,

H-Asn-(Glu)5des Pro36, Pro37, Pro38 [Asp28] Exendin-4(1-39)-NH2,

des Pro36, Pro37, Pro38 [Asp28] Exendin-4(1-39)-(Lys)6-NH2,

H-(Lys)6-des Pro36, Pro37, Pro38 [Asp28] Exendin-4(1-39)-(Lys)6-NH2,

H-Asn-(Glu)5-des Pro36, Pro37, Pro38 [Asp28] Exendin-4(1-39)-(Lys)6-NH2,

H-(Lys)6-des Pro36 [Trp(O2)25, Asp28] Exendin-4(1-39)-Lys6-NH2,

H-des Asp28 Pro36, Pro37, Pro38 [Trp(O2)25] Exendin-4(1-39)-NH2,

H-(Lys)6-des Pro36, Pro37, Pro38 [Trp(O2)25, Asp28] Exendin-4(1-39)-NH2,

H-Asn-(Glu)5-des Pro36, Pro37, Pro38 [Trp(O2)25, Asp28] Exendin-4(1-39)-NH2,

des Pro36, Pro37, Pro38 [Trp(O2)25, Asp28] Exendin-4(1-39)-(Lys)6-NH2,

H-(Lys)6-des Pro36, Pro37, Pro38 [Trp(O2)25, Asp28] Exendin-4(1-39)-(Lys)6-NH2,

H-Asn-(Glu)5-des Pro36, Pro37, Pro38 [Trp(O2)25, Asp28] Exendin-4(1-39)-(Lys)6-NH2,

H-(Lys)6-des Pro36 [Met(O)14, Asp28] Exendin-4(1-39)-Lys6-NH2,

des Met(O)14 Asp28 Pro36, Pro37, Pro38 Exendin-4(1-39)-NH2,

H-(Lys)6-desPro36, Pro37, Pro38 [Met(O)14, Asp28] Exendin-4(1-39)-NH2,

H-Asn-(Glu)5-des Pro36, Pro37, Pro38 [Met(O)14, Asp28] Exendin-4(1-39)-NH2,

des Pro36, Pro37, Pro38 [Met(O)14, Asp28] Exendin-4(1-39)-(Lys)6-NH2,

H-(Lys)6-des Pro36, Pro37, Pro38 [Met(O)14, Asp28] Exendin-4(1-39)-(Lys)6-NH2,

H-Asn-(Glu)5 des Pro36, Pro37, Pro38 [Met(O)14, Asp28] Exendin-4(1-39)-(Lys)6-NH2,

H-Lys6-des Pro36 [Met(O)14, Trp(O2)25, Asp28] Exendin-4(1-39)-Lys6-NH2,

H-des Asp28 Pro36, Pro37, Pro38 [Met(O)14, Trp(O2)25] Exendin-4(1-39)-NH2,

H-(Lys)6-des Pro36, Pro37, Pro38 [Met(O)14, Asp28] Exendin-4(1-39)-NH2,

H-Asn-(Glu)5-des Pro36, Pro37, Pro38 [Met(O)14, Trp(O2)25, Asp28] Exendin-4(1-39)-NH2,

des Pro36, Pro37, Pro38 [Met(O)14, Trp(O2)25, Asp28] Exendin-4(1-39)-(Lys)6-NH2,

H-(Lys)6-des Pro36, Pro37, Pro38 [Met(O)14, Trp(O2)25, Asp28] Exendin-4(S1-39)-(Lys)6-NH2,

H-Asn-(Glu)5-des Pro36, Pro37, Pro38 [Met(O)14, Trp(O2)25, Asp28] Exendin-4(1-39)-(Lys)6-NH2;

or a pharmaceutically acceptable salt or solvate of any one of the afore-mentioned Exendin-4 derivative.

Hormones are for example hypophysis hormones or hypothalamus hormones or regulatory active peptides and their antagonists as listed in Rote Liste, ed. 2008, Chapter 50, such as Gonadotropine (Follitropin, Lutropin, Choriongonadotropin, Menotropin), Somatropine (Somatropin), Desmopressin, Terlipressin, Gonadorelin, Triptorelin, Leuprorelin, Buserelin, Nafarelin, Goserelin.

A polysaccharide is for example a glucosaminoglycane, a hyaluronic acid, a heparin, a low molecular weight heparin or an ultra low molecular weight heparin or a derivative thereof, or a sulphated, e.g. a poly-sulphated form of the above-mentioned polysaccharides, and/or a pharmaceutically acceptable salt thereof. An example of a pharmaceutically acceptable salt of a poly-sulphated low molecular weight heparin is enoxaparin sodium.

Antibodies are globular plasma proteins (−150 kDa) that are also known as immunoglobulins which share a basic structure. As they have sugar chains added to amino acid residues, they are glycoproteins. The basic functional unit of each antibody is an immunoglobulin (Ig) monomer (containing only one Ig unit); secreted antibodies can also be dimeric with two Ig units as with IgA, tetrameric with four Ig units like teleost fish IgM, or pentameric with five Ig units, like mammalian IgM.

The Ig monomer is a “Y”-shaped molecule that consists of four polypeptide chains; two identical heavy chains and two identical light chains connected by disulfide bonds between cysteine residues. Each heavy chain is about 440 amino acids long; each light chain is about 220 amino acids long. Heavy and light chains each contain intrachain disulfide bonds which stabilize their folding. Each chain is composed of structural domains called Ig domains. These domains contain about 70-110 amino acids and are classified into different categories (for example, variable or V, and constant or C) according to their size and function. They have a characteristic immunoglobulin fold in which two β sheets create a “sandwich” shape, held together by interactions between conserved cysteines and other charged amino acids.

There are five types of mammalian Ig heavy chain denoted by α, δ, ε, γ, and μ. The type of heavy chain present defines the isotype of antibody; these chains are found in IgA, IgD, IgE, IgG, and IgM antibodies, respectively.

Distinct heavy chains differ in size and composition; α and γ contain approximately 450 amino acids and δ approximately 500 amino acids, while μ and ε have approximately 550 amino acids. Each heavy chain has two regions, the constant region (CH) and the variable region (VH). In one species, the constant region is essentially identical in all antibodies of the same isotype, but differs in antibodies of different isotypes. Heavy chains γ, α and δ have a constant region composed of three tandem Ig domains, and a hinge region for added flexibility; heavy chains μ and ε have a constant region composed of four immunoglobulin domains. The variable region of the heavy chain differs in antibodies produced by different B cells, but is the same for all antibodies produced by a single B cell or B cell clone. The variable region of each heavy chain is approximately 110 amino acids long and is composed of a single Ig domain.

In mammals, there are two types of immunoglobulin light chain denoted by λ and κ. A light chain has two successive domains: one constant domain (CL) and one variable domain (VL). The approximate length of a light chain is 211 to 217 amino acids. Each antibody contains two light chains that are always identical; only one type of light chain, κ or λ, is present per antibody in mammals.

Although the general structure of all antibodies is very similar, the unique property of a given antibody is determined by the variable (V) regions, as detailed above. More specifically, variable loops, three each the light (VL) and three on the heavy (VH) chain, are responsible for binding to the antigen, i.e. for its antigen specificity. These loops are referred to as the Complementarity Determining Regions (CDRs). Because CDRs from both VH and VL domains contribute to the antigen-binding site, it is the combination of the heavy and the light chains, and not either alone, that determines the final antigen specificity.

An “antibody fragment” contains at least one antigen binding fragment as defined above, and exhibits essentially the same function and specificity as the complete antibody of which the fragment is derived from. Limited proteolytic digestion with papain cleaves the Ig prototype into three fragments. Two identical amino terminal fragments, each containing one entire L chain and about half an H chain, are the antigen binding fragments (Fab). The third fragment, similar in size but containing the carboxyl terminal half of both heavy chains with their interchain disulfide bond, is the crystalizable fragment (Fc). The Fc contains carbohydrates, complement-binding, and FcR-binding sites. Limited pepsin digestion yields a single F(ab′)2 fragment containing both Fab pieces and the hinge region, including the H-H interchain disulfide bond. F(ab′)2 is divalent for antigen binding. The disulfide bond of F(ab′)2 may be cleaved in order to obtain Fab′. Moreover, the variable regions of the heavy and light chains can be fused together to form a single chain variable fragment (scFv).

Pharmaceutically acceptable salts are for example acid addition salts and basic salts. Acid addition salts are e.g. HCl or HBr salts. Basic salts are e.g. salts having a cation selected from alkali or alkaline, e.g. Na+, or K+, or Ca2+, or an ammonium ion N+(R1)(R2)(R3)(R4), wherein R1 to R4 independently of each other mean: hydrogen, an optionally substituted C1 C6-alkyl group, an optionally substituted C2-C6-alkenyl group, an optionally substituted C6-C10-aryl group, or an optionally substituted C6-C10-heteroaryl group. Further examples of pharmaceutically acceptable salts are described in “Remington's Pharmaceutical Sciences” 17. ed. Alfonso R. Gennaro (E d.), Mark Publishing Company, Easton, Pa., U.S.A., 1985 and in Encyclopedia of Pharmaceutical Technology.

Pharmaceutically acceptable solvates are for example hydrates.

Preferably, the piston rod is axially moved and rotated in the delivery direction. Alternatively, rotational movement of the piston rod may be prevented such that the piston rod may be moved only axially during dose delivery. The piston rod may be prevented from any movement during a dose setting operation of the assembly. The assembly may further comprise an energy storing member, e.g. a spring member. The energy storing member may be a torsion spring, for example. The energy storing member may be adapted and arranged to store energy. The energy storing member may be adapted and arranged for moving the piston rod in the delivery direction. In particular, the piston rod may be moved by the energy storing member, in particular by means of energy stored in the energy storing member, for delivering a dose of the drug. Energy may be released from the energy storing member when an activation member, e.g. a dose button, is operated by a user. The released energy may cause the piston rod to be moved in the delivery direction.

The assembly further comprises an interaction member. The interaction member may be adapted and arranged to mechanically cooperate with the piston rod. The interaction member is dispaceable, for example tiltable or rotatable or shiftable, between a first position and a second position with respect to the piston rod. The interaction member may be configured to be moved from the first position towards the second position to increase a friction onto the piston rod. The interaction member may further be configured to be moved from the second position back into the first position to decrease the friction onto the piston rod.

The amount of friction generated is increased when the interaction member is moved towards the second position. Friction will impact the movement of the piston rod such that the piston rod becomes slower. Further movement of the interaction member towards the second position and, thus, more friction will further reduce the speed of the piston rod. Varying the position of the interaction member thus leads to a variation of the friction exerted onto the piston rod and, therefore, allows controlling the speed of movement of the piston rod. In this way, provision of a drug delivery device comprising high user comfort is facilitated.

According to an embodiment, when the interaction member is positioned in the first position, the piston rod is displaceable in the delivery direction at a first speed. When the interaction member is moved towards the second position, the interation member may generate a force, e.g. a radial force or a twisted tension, onto the piston rod. The more the interaction member is moved towards the second position, the greater the force. The piston rod may be displaceable in the delivery direction at a second speed when the interaction member is moved towards the second position. The second speed may be smaller than the first speed.

The amount of the force generated and, thus, of the friction, depends on the position of the interaction member with respect to the piston rod. Thus, for achieving more friction and, thus, a lower speed of the piston rod, the interaction member must be moved further away from the first position and into the second position. In this way, a number of different speeds of the piston rod can be achieved by means of varying the position of the interaction member. In particular, the piston rod can be slowed down by means of moving the interaction member away from the first position. The interaction member acts as a brake for the piston rod by means of which the injection speed of the piston rod can be adjusted to the individual need of a user. In this way, provision of a device with high user comfort is facilitated.

According to an embodiment, the interaction member may be a lever disc. The interaction member may comprise a first portion. The first portion may be shaped disc-like. The interaction member may comprise a second portion. The second portion may be an arm portion or a handle portion. The first and second portion may together form the interaction member. Preferably, the interaction member is formed unitarily.

The first portion may be arranged around the piston rod. In particular, the piston rod may be guided through the first portion. The interaction member and, in particular, the first portion may have an opening, in particular a through-going opening, surrounding the piston rod. During dose delivery, the piston rod may be moved in the delivery direction through the opening. A surface of the first portion which faces the piston rod may comprise friction increasing elements. For example, the surface of the opening may comprise a thread and/or the piston rod may comprise an outer thread. Alternatively, the opening may comprise a rough surface and/or may be oriented such that a contact area between the interaction member and the piston rod may be maximized when the interaction member is in the second position.

The second portion may comprise an elongated shape. The second portion may protrude from the first portion in a radial direction. The second portion may be configured to be operated by the user. The assembly may further comprise a housing. The housing may comprise an opening. The second portion may be adapted and arranged to extend through the opening of the housing. The second portion may protrude from the housing in a radial direction. By means of operating the second portion, a user can control the position of the interaction member. Accordingly, by means of operating the second portion, the user can individually control the speed of movement of the piston rod, i.e. the injection speed. Hence, provision of a device with increased user comfort is facilitated.

According to an embodiment, the second portion is biased towards the first position or towards the second position. The second portion may be biased by a spring member into the first position. This may allow movement of the piston rod during dose delivery at a full speed without further intervention by the user. The user may then control the speed by moving the interaction member towards the second position. The user may move the interaction member, in particular the second portion, wherein movement of the second portion controls the injection speed, i.e. the speed of movement, of the piston rod.

Alternatively, the second portion may be biased in the opposite direction, i.e. towards the second position, thus allowing to control the speed of the piston rod in an opposite manner. Moving the second portion against the bias, i.e. towards the first position, would in this scenario increase the speed of the piston rod.

According to an embodiment, the piston rod is in direct mechanical contact with the interaction member. The piston rod may be in direct mechanical contact with the interaction member, in particular with the first portion, at least when the interation member is arranged in the second position. When the interaction member is moved from the first position towards the second position, a contact area between the interaction member and the piston rod may be increased. The contact area may be maximized when the interaction member is in the second position. The contact area may be minimized when the interaction member is in the first position. Accordingly, by moving the interaction member between the first and the second position, the contact area and, thus, the friction onto the piston rod may be varied. In the second position, an inner edge of the first portion may be at least partly in contact with the piston rod, thereby generating friction. In the second position, the interaction member and the piston rod may engage one another.

In an alternative embodiment, the piston rod and the interaction member may be engaged throughout the operation of the device, irrespective of whether the interaction member is arranged in the first or in the second position. When the interaction member is arranged in the first position, the contact area between the piston rod and the interaction member may be smaller than the contact area when the interaction member is arranged in the second position. When the interaction member and the piston rod engage, i.e. when the contact area is greater than zero, when the interaction member is in the first position, the opening of the interaction member has to be configured to provide sufficient play such that the piston rod is moveable in the delivery direction during a dose delivery operation.

According to an embodiment, for moving the interaction member from the first position into the second position, the interaction member is tiltable about an axis which is perpendicular to an axis along which the piston rod is moved during a dose delivery operation. The interaction member may comprise the previously mentioned opening. The piston rod is guided through the opening. When the interaction member is arranged in the first position, a surface normal of the opening may be parallel to the axis which is perpendicular to the axis along which the piston rod is moved during a dose delivery operation. The contact area between the interaction member and the piston rod may be minimized in this position.

When the interaction meber is moved from the first position towards the second position the surface normal is moved by an angle. Varying said angle may influence the amount of contact between the interaction member and the piston rod. Varying said angle may cause varying the friction and, therefore, allows to control the speed of the piston rod. In particular, an amount of friction onto the piston rod may depend on the size of the angle between the surface normal of the opening and the axis perpendicular to the axis along which the piston rod is moved during a dose delivery operation. The amount of friction generated is increased when the interaction member is tilted further. Friction will impact the movement of the piston rod such that the piston rod becomes slower. More friction will, thus, further reduce the speed of the piston rod. When the interaction member is arranged in the second position, the surface normal of the opening may be arranged at a specific angle with respect to the axis which is perpendicular to the axis along which the piston rod is moved during a dose delivery operation. The angle may be unequal to zero. The angle may be between 1 and 45 degree, for example. The angle may amount to 5, 10 or 15 degree, for example.

According to an embodiment, the assembly further comprises a guide member. The guide member may be a guide nut. The guide member may be a thread nut. The guide member is adapted and arranged to mechanically cooperate with the piston rod to guide movement of the piston rod in the delivery direction. Mechanical cooperation between the guide member and the piston rod may result in a rotational movement of the piston rod in the delivery direction. In particular, the piston rod may perform a combined translational and rotational movement during dose delivery. For this purpose, the piston rod may comprise a thread. The opening of the interaction member may comprise a thread that substantially conforms to the thread of the piston rod.

When the interaction member is positioned in the first position, it has a predetermined first distance with respect to the guide member. In the first position the interaction member is preferably not in contact with the guide member. The piston rod can rotate through the opening of the interaction member and of the guide member for dose delivery. The interaction member may be rotatable about an angle towards the guide member into the second position.

According to an embodiment, when the interation member is moved from the first position into the second position, the interaction member is rotated towards the guide member along the axis along which the piston rod is moved during a dose delivery operation. The interaction member may be rotated by an angle. The interaction member may be configured to be rotated about the axis between the first position and the second position.The interaction member may be rotated such that the interaction member is arranged at a second distance with respect to the guide member when the interaction member is positioned in the second position. The second distance may be smaller than the first distance.

When the interaction member is arranged in the second position, the interaction member may abut the guide member to increase the friction. In the second position the interaction member may work like a counter nut or jam nut. In the second position the interaction member engages with the piston rod such that friction is generated. An amount of friction onto the piston rod may depend on the angle by which the interaction member is rotated towards the guide member. Hence, determining the angle of rotation of the interaction member allows controlling the movement of the piston rod. Varying the angle causes varying the friction and, therefore, allows controlling the speed of movement of the piston rod. The user can individually control the position of the interaction member and, thus, the speed of the piston rod during dose delivery by means of rotating the second portion of the interaction member. In this way provision of a drug delivery device with high user comfort is facilitated.

A further aspect relates to a drug delivery device. The device may comprise the previously mentioned assembly. In particular, the assembly may be implemented in the device. The device may further comprise a cartridge for holding a plurality of doses of the drug. The device or the assembly may further comprise an activation member adapted and arranged to be operated by a user for triggering a dose delivery operation of the assembly. The activation member is axially displaceable in the delivery direction for delivering a dose of the drug. The axial direction may be the direction along a main longitudinal axis of the assembly and, thus, of the device. The activation member is prevented from being rotated or tilted with respect to the housing for delivering a dose of the drug. The activation member is prevented from being moved in a radial direction with respect to a main longitudinal axis of the assembly and, thus, of the device for delivering a dose of the drug.

The device may further comprise the previously mentioned energy storing member. The energy storing member is adapted and arranged to store energy during a dose setting operation. The energy storing member is adapted and arranged to move the piston rod in the delivery direction when the activation member is operated by the user. When the activation member is operated, the piston rod may be driven due to the energy stored in the energy storing member for delivering the dose. In particular, the activation member is configured to release the energy stored in the energy storing member. The device may be an automatic device. By means of the assembly, the user can control the speed of movement of the piston rod. In particular, he can adjust the speed to his individual needs. Accordingly, a very user friendly device is provided.

According to a further aspect, the previously mentioned assembly may be operated such that an interaction member is moved, wherein movement of the interaction member controls a speed of movement of a piston rod. The interaction member may be moved, e.g. rotated, tilted or shifted, between a first position and a second position. Operating the assembly may comprise the step of moving the interaction member in a first direction, wherein friction between the interaction member and the piston rod is increased. Operating the assembly may further comprise the step of moving the interaction member in a second direction, wherein friction between the interaction member and the piston rod is decreased. The second direction may be opposite to the first direction. Increasing and decreasing the friction may allow to control the speed of movement of the piston rod and, thereby, allows to control of dispense of a drug from a cartridge.

Of course, features described above in connection with different aspects and embodiments may be combined with each other and with features described below.

Further features and refinements become apparent from the following description of the exemplary embodiments in connection with the accompanying figures.

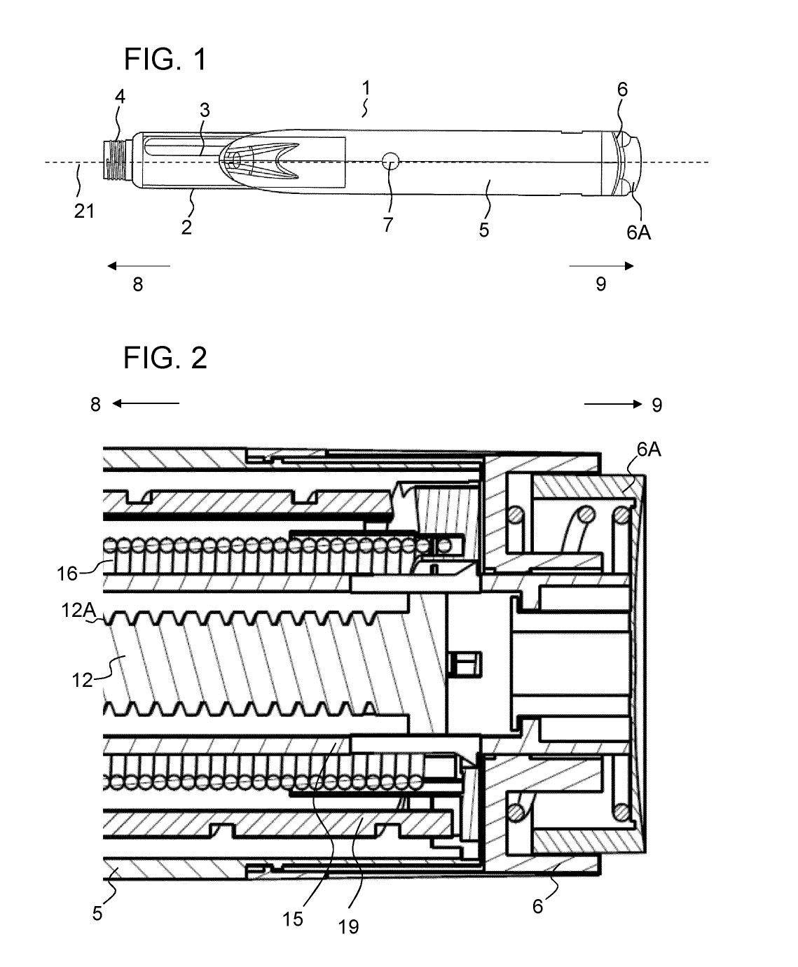

FIG. 1 schematically shows a three-dimensional view of a drug delivery device,

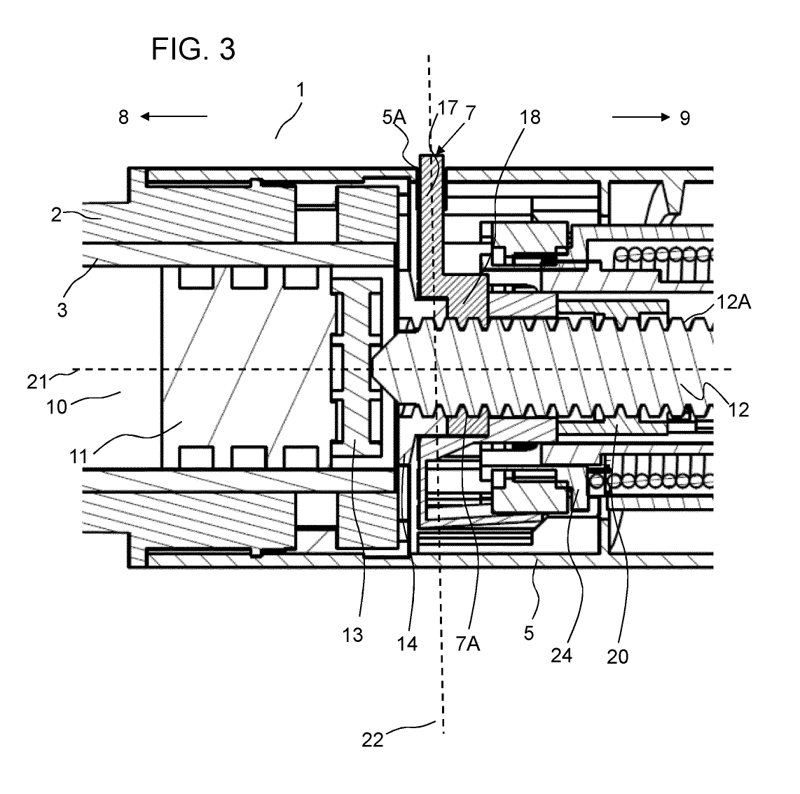

FIG. 2 schematically shows a sectional side view of a part of the drug delivery device of FIG. 1,

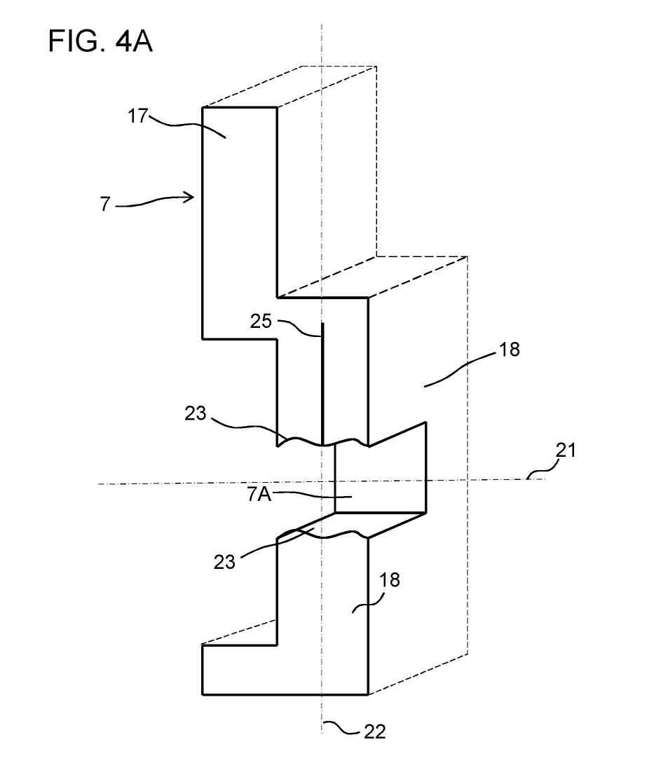

FIG. 3 schematically shows a sectional side view of a part of the drug delivery device of FIG. 1 according to one embodiment,

FIGS. schematically 4A and 4B show a sectional view of a part of the drug delivery device of FIG. 1 according to another embodiment.

Like elements, elements of the same kind and identically acting elements may be provided with the same reference numerals in the figures.

In FIG. 1 a drug delivery device 1 is shown. The drug delivery device 1 comprises a housing 5. The housing 5 is adapted and arranged for protecting components of the device 1 arranged within the housing 5 from environmental influences. The drug delivery device 1 and the housing 5 have a distal end 8 and a proximal end 9. The term “distal end” designates that end of the drug delivery device 1 or a component thereof which is or is to be arranged closest to a dispensing end of the drug delivery device 1. The term “proximal end” designates that end of the device 1 or a component thereof which is or is to be arranged furthest away from the dispensing end of the device 1. The distal end 8 and the proximal end 9 are spaced apart from one another in the direction of an axis 21. The axis 21 may be the longitudinal axis or rotational axis of the device 1.

The drug delivery device 1 comprises a cartridge holder 2. The cartridge holder 2 comprises a cartridge 3. The cartridge 3 contains a drug 10, preferably a plurality of doses of the drug 10. The cartridge 3 is retained within the cartridge holder 2. The cartridge holder 2 stabilizes the position of the cartridge 3 mechanically. The cartridge holder 2 is connectable, e.g. by a threaded engagement or by a bayonet coupling, to the housing 5. The cartridge holder 2 and the housing 5 may be releasably or non-releasably connected to one another. In an alternative embodiment, the cartridge 3 may be directly connected to the housing 5. In this case, the cartridge holder 2 may be redundant.

The drug delivery device 1 may be a pen-type device, in particular a pen-type injector. The device 1 may be a re-usable device, which means that the cartridge 3 can be replaced, in particular during a reset operation, by a replacement cartridge for dispensing a plurality of doses from the replacement cartridge. Alternatively, the device 1 may be a disposable device. The device 1 can be configured to dispense variable doses of the drug 10. Alternatively, the device 1 may be a fixed dose device. The device 1 may be an auto-injection device. This means that a dose delivery operation of the device 1 may be initiated by the user by pressing an actuation member 6A, wherein upon pressing the actuation member 6A an energy stored in the device 1 is released for dispensing a dose of the drug 10. The auto-injector may be configured to substantially expel the entire content from the cartridge 3. Alternatively, the auto-injector may comprise a dose setting member configured to determine the amount of drug 10 to be expelled.

A bung 11 (see FIG. 3) is slideably retained within the cartridge 3. The bung 11 seals the cartridge 3 proximally. Movement of the bung 11 in the distal direction with respect to the cartridge 3 causes the drug 10 to be dispensed from the cartridge 3. A needle assembly (not explicitly shown in the Figures) can be arranged at the distal end section of the cartridge holder 2, e.g. by means of an engagement means 4, e.g. a thread.

FIGS. 2 and 3 show a sectional side view of a part of the drug delivery device of FIG. 1.

The device 1 comprises a piston rod 12, which is configured to be moved in a distal or delivery direction in order to dispense a dose of the drug 10. The piston rod 12 is moved along the longitudinal axis 21 of the device 1. The longitudinal axis 21 is, thus, the axis of movement of the piston rod 12. The piston rod 12 is configured to move the bung 11 arranged in the cartridge 3 towards the dispensing end of the drug delivery device 1. The piston rod 12 comprises a bearing member 13 which is in contact with the bung 11. The piston rod 12 is configured as a lead screw. The piston rod 12 comprises an outer thread 12A. The thread 12A is arranged along an outer surface of the piston rod 12. The device 1 further comprises a drive member 20. The drive member 20 is configured as a spline nut. The drive member 20 is engaged with the piston rod 12. In particular, the drive member 20 comprises splines, which are engaged with axial grooves of the piston rod 12. Thus, the drive member 20 is rotationally fixed but axially moveable with respect to the piston rod 12. The drive member 20 and, thus, the piston rod 12 may be rotatable during a dose delivery operation.

The device 1 may further comprise a guide member 14. The piston rod 12 is guided through the guide member 14. The guide member 14 is arranged around the piston rod 12. The guide member 14 may be a nut member. The guide member 14 is preferably configured as a thread nut. The guide member 14 is in threaded engagement, preferably in permanent threaded engagement, with the piston rod 12. For this purpose, the guide member 14 comprises an inner thread which is in engagement with the outer thread 12A of the piston rod 12. The guide member 14 is secured against movement with respect to the housing 5. Due to the threaded engagement between the piston rod 12 and the guide member 14, rotational movement of the piston rod 12 in the distal or delivery direction is enabled. Thereby, the bung 11 is moved in the distal direction for dispensing a dose of the drug 10. In an alternative embodiment (not explicitly shown), the piston rod 12 may be prevented from rotation with respect to the housing 5. In this embodiment, the delivery movement of the piston rod 12 may be an axial movement in the distal direction. In this case, an outer thread of the piston rod 12 may be redundant.

Prior to dose delivery, the required dose of drug 10 is set by means of a dose setting mechanism. Common designs of dose setting mechanisms comprise a number of tubular or sleeve-like members such as a dose setting sleeve, a dose indicating sleeve, a drive sleeve and/or a ratchet sleeve. Such sleeves are often accommodated within and connected to each other.

The device 1 and, in particular the dose setting mechanism, comprises a dose setting member 6. The dose setting member 6 may be shaped sleeve-like. The dose setting member 6 is configured for setting a dose of the drug 10. The dose setting member 6 can be rotated by a user for setting a dose. The dose setting member 6 is axially fixed with respect to the housing 5. The dose setting member 6 is prevented from rotation during a dose delivery operation. The device 1 further comprises a drive shaft 15. By rotating the dose setting member 6 during a dose setting operation, the drive shaft 15 is also rotated. In particular, the drive shaft 15 may be rotationally fixed to the dose setting member 6 during the dose setting operation due to a splined connection.

The device 1 furthermore comprises a rotation member 19. The rotation member 19 is configured as a sleeve. The rotation member 19 is arranged concentrically around the drive shaft 15. The rotation member 19 may be fixed to the drive shaft 15, e.g. by a snap-fit connection. The rotation member 19 is axially fixed with respect to the drive shaft 15. Rotation of the drive shaft 15 during a dose setting operation causes rotation of the rotation member 19. The direction in which the drive shaft 15 and the rotation member 19 are rotated during dose setting, i.e. a first rotational direction, may be a clockwise direction.

To improve the user comfort the device 1 comprises a power assistance, in particular an energy storing member 16. The energy storing member 16 may be a coil spring. The energy storing member 16 may be a torsion spring. When the rotation member 19 is rotated during dose setting, the energy storing member 16 is compressed, such that energy is stored in the energy storing member 16. Thereby, a clicking noise is produced e.g. by a single ratchet on a ratchet sleeve (not explicitly shown in the Figures).

The device 1 further comprises a locking member 24. The locking member 24 is rotationally fixed with respect to the housing 5 during the setting of a dose, e.g. by means of a splined connection. The rotation member 19 is engaged with the locking member 24, e.g. by a toothed connection such that the rotation of the rotation member 19 in the first roational direction is allowed during the dose setting operation.

The device 1 further comprises the previously mentioned actuation member 6A. In order to dispense a dose, the actuation member 6A is operated by a user. The actuation member 6A may comprise a button. Once pressing the actuation member 6A, the energy storing member 16 is released and drives the piston rod 12 to deliver a dose of the drug 10 from the cartridge 3. The user of the device 1 does, thus, not have to provide the force for dispense.

In particular, when the actuation member 6A is actuated, e.g. moved in a distal direction, the drive shaft 15 is also moved in a distal direction. The drive shaft 15 is thereby moved out of engagament with the dose setting member 6. When the drive shaft 15 is moved in the distal direction, the rotation member 19 and the locking member 24 are also moved in the distal direction. Thereby, the locking member 24 is disengaged from its engagement with the housing 5. In particular, the locking member 24 is allowed to rotate with respect to the housing 5 when the actuation member 6A is actuated by a user. When the locking member 24 is allowed to rotate, the rotation member 19 is allowed to rotate, as well. When the locking member 24 is enabled to rotate with respect to the housing 5, the energy which is stored in the energy storing member 16 is released causing a rotation of the rotation member 19. Rotation of the rotation member 19 causes rotation of the drive member 20 and, thus, rotational movement of the piston rod 12 in the distal direction for dispensing a dose of the drug 10. In an alternative embodiment (not explicitly shown), the device 1 may be configured such that operation of the activation member 6A results in an axial movement of the piston rod 12 without being rotated with respect to the housing 5 as already mentioned above.

In conventional drug delivery devices, the user has no control over the speed with which the piston rod 12 is moved during the dose delivery operation, i.e. the injection speed. In particular, when the above mentioned locking member 24 is released, the injection speed is independent from the pressed actuation member 6A. However, it is desirable for the user to control the injection speed, e.g. to reduce the pain when the drug 10 is injected into the tissue.

For this purpose, the device comprises an interaction member 7. The interaction member 7 comprises a first portion 18. The first portion 18 is formed disc-like. The interaction member 7 comprises a second portion 17. The second portion 17 may be an arm part. The second portion 17 is adapted and arranged to be moved by the user. The second portion 17 protrudes from the housing 5 of the device 1 as can be seen from FIG. 3. For this purpose, the housing 5 comprises an opening 5A. The second portion 17 protrudes in the radial outward direction from the first portion 18 and through the opening 5A of the housing 5. Thus, the interaction member 7 can be directly accessed by the user form an outside of the housing 5. In particular, the user can access the second portion 17 and use the second portion 17 as a handle. The interaction member 7 may be formed unitarily. In an alternative embodiment (not explicitly shown) first and second portion 17, 18 may be formed separately and, thus, may be connected to one another to form the interaction member 17.

The interaction member 7 is configured to mechanically cooperate with the piston rod 12. The interaction member 7 is arranged around the piston rod 12. In particular, the piston rod 12 is guided through the interaction member 7. For this purpose the interaction member 7 and, in particular the first portion 18, comprises an opening 7A. The opening 7A may be a thread hole. In other words a surface confining the opening 7A can comprise a thread. The thread of the opening 7A may comprise the same pitch as the thread 12A of the piston rod 12 and as the thread of the guide member 14. However, the thread play between the interaction member 7 and the piston rod 12 is preferably bigger than the thread play between the piston rod 12 and the guide member 14. In this way, the interaction member 7 may be moveable with respect to the piston rod 12. In an alternative embodiment (not explicitly shown), the surface 23 of the opening 7A may be free from a thread. The opening 7A comprises a surface normal 25. The surface normal 25 is a line which is perpendicular to the surface 23 of the opening 7A. The surface 23 confines the opening 7A. The surface 23 of the opening 7A may be that surface of the interaction member 7 which faces the outer surface of the piston rod 12 (see FIG. 3).

The interaction member 7 is moveable, e.g. tiltable, rotatable or shiftable. In particular, the interaction member 7 is moveable between a first position and a second position with respect to the piston rod 12. The interaction member 7 is configured to be moved from the first position towards the second position to increase a friction onto the piston rod 12, e.g. by means of increasing or establishing a contact area between the piston rod 12 and the interaction member 7. The interaction member 7 is configured to be moved from the second position back into the first position to decrease the friction onto the piston rod 12, e.g. by decreasing or removing the contact area between the piston rod 12 and the interaction member 7.

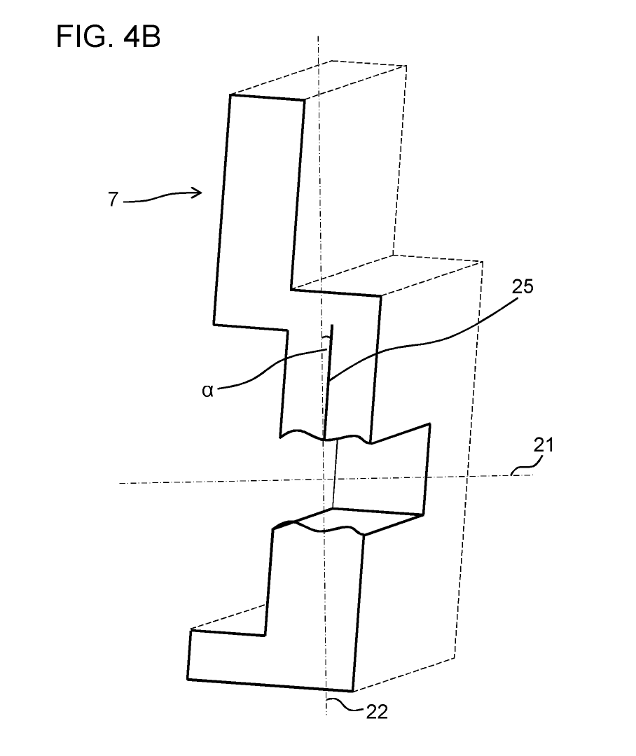

According to one embodiment (see FIGS. 4A and 4B), for being moved between the first position and the second position the interaction member 7 is tilted about an axis 22. The axis 22 is an axis perpendicular to the longitudinal axis 21 of the device 1. The longitudinal axis 21 is the axis along which the piston rod 12 is moved during dose delivery. The interaction member 7 is tilted by means of the user moving the second portion 18, i.e. the arm portion 18 about the axis 22. When the interaction member 7 is arranged in the first position (see FIG. 4A), the surface normal 25 of the opening 7A is parallel to the axis 22 which is perpendicular to the longitudinal axis 21 of the device 1. In other words, the surface normal 25 of the opening 7A is perpendicular to the longitudinal axis 21. Thus, the surface 23 of the opening 7A is parallel to the outer surface of the piston rod 12.

In the first position, there must not necessarily be mechanical cooperation let alone threaded engagement between the piston rod 12 and the interaction member 7. The opening 7A may have a diameter such that the piston rod 12 can be guided through the opening 7A without mechanical cooperation with the surface of the opening 7A when the interaction member 7 is in the first position. In this case, the contact area between the piston rod 12 and the interaction member 7 is equal to zero. Thus, in this embodiment, there is no friction at all between the piston rod 12 and the interaction member 7 when the interaction member 7 is in the first position.

Alternatively, the interaction member 7 may engage the piston rod 12 also when the interaction member 7 is arranged in the first position. In this case, the contact area may be greater than zero. However, when the interaction member 7 is arranged in the first position, the contact area between the piston rod 12 and the interaction member 7 may be smaller than the contact area when the interaction member 7 is arranged in the second position. When the interaction member 7 and the piston rod 12 engage, i.e. when the contact area is greater than zero, when the interaction member 7 is in the first position, the opening 7A has to be configured to provide sufficient play in the threading connection to allow a tilting movement of the interaction member 7. In other words, the opening 7A has to be configured to provide sufficient play in the threading connection to allow movement of the interaction member about the axis 22.

When the interaction member 7 is arranged in the second position, an inner edge of the surface of the opening 7A is least partly in contact with the piston rod 12, thereby generating friction. Accordingly, when the interaction member 7 is arranged in the second position, the interaction member 7 always engages the piston rod 12. When the interaction member 7 is arranged in the second position, the interation member 7 exerts a radial force onto the piston rod 12. When the interaction member 7 is arranged in the second position, the contact area between the piston rod 12 and the interaction member 7 is increased as compared to the contact area when the interaction member 7 is in the first position. When the interaction member 7 is moved towards the second position (see FIG. 4B), the surface normal 25 of the opening 7A is moved by an angle α. When the interaction member 7 is moved towards the second position, the contact area between the interaction member 7 and the piston rod 12 is continuously increased. Thus, when the interaction member 7 is moved towards the second position the radial force exterted by the interaction member 7 onto the piston rod 12 is increased.

When the interaction member 7 is positioned in the second position (see FIG. 4B), the surface normal 25 of the opening 7A is arranged at the angle α with respect to the axis 22. The angle α is greater than zero. The angle α between the surface normal 15 of the opening 7A and the axis 22 may be between 1 and 45 degree. The angle α may be smaller than 30 degree, for example 15, 10 or 5 degree. The angle α is indicative of the position of the interaction member 7 with respect to the piston rod 12. Different positions of the interaction member 7 may correspond to different angels. Varying the angle α by tilting the second portion 17 around the axis 22 causes varying the position of the interaction member 7. Varying the angle α causes varying the contact area and, thus, the friction between the piston rod 12 and the interaction member 7, thus allowing to control the speed of movement of the piston rod 12. The amount of friction generated is increased when the interaction member 7 is tilted further. Accordingly, the greater the angle α between the surface normal 25 of the opening 7A and the axis 22, the greater the friction between the piston rod 12 and the interaction member 7.

Friction will impact the movement of the piston rod 12 such that the piston rod 12 becomes slower. More friction will further reduce the speed of the piston rod 12. Varying the angle between the surface normal 25 of the opening 7A and the axis 22 causes varying the friction and therefore allows controlling the speed of movement of the piston rod 12. In particular, when the interaction member 7 is arranged in the first position, the angle α between the surface normal 25 of the opening 7A and the axis 22 preferably amounts to 0 degree (see FIG. 4A). When the interaction member 7 is arranged in the first position, the piston rod 12 is, thus, displaceable in the delivery direction (i.e. the distal direction) at a first speed. When the interaction member is arranged in the second position (see FIG. 4B), the angle α between the surface normal 25 of the opening 7A and the axis 22 is greater than 0 degree. Thus, the piston rod 12 is displaceable in the delivery direction at a second speed. The second speed is smaller than the first speed. Thus, by means of the interaction member 7, the injection speed can be controlled and, in particular, reduced by the user.

In this embodiment, the operation of the interaction member 7 is independent from the function of the guide member 14. Thus, in this embodiment, a guide member 14 is not necessarily needed, e.g. for achieving a rotational delivery movement of the piston rod 12. Thus, in this embodiment, the delivery movement of the piston rod 12 may be linear movement or a combined linear and rotational movement.

The dose setting operation is not influenced by the operation of the interaction member 7 as the piston rod 12 is not moveable during the dose setting operation but only during the dose delivery operation.

The second portion 18 can be biased into the first position or into the second position, e.g. by means of a spring member (not explicitly shown in the Figures). The second portion 18 may be biased by a spring into the first position. This allows movement of the piston rod 12 during the dose delivery operation at full speed without further intervention by the user. The user can then control the speed by moving the second portion 18 and, thus, the interaction member 7 into the second position as described above. Alternatively, the second portion 18 may be biased in the opposite direction, i.e. into the second position, thus allowing controlling speed in an opposite manner. Moving the second portion 18 against the bias will, thus, increase the injection speed. In an alternative embodiment, the second portion 18 is not biased at all. In this embodiment, the user can adjust the interaction member 7 and, in particular the position of the interaction member 7 with respect to the piston rod 12 once and the injection speed will stay the same throughout the lifetime of the device 1.

The opening 7A of the interaction member 7 may comprise a rough surface 23 to increase the friction between the interaction member 7 and the piston rod 12 when the interaction member 7 is arranged in the second position. The interaction member 7 may comprise an elastic material. The interaction member 7 may, for example, comprise rubber. In this way, a friction between the interaction member 7 and the piston rod 12 may be increased. Alternatively, the interaction member 7 may be formed from a non-elastic material, e.g. from plastic.

In an alternative embodiment (see FIG. 3), the interaction member 7 is configured to be rotated about the longitudinal axis 22. In particular, the interaction member 7 is configured to be rotated about the axis 22 along which the piston rod 12 is moved during dose delivery. The interaction member 7 is rotatable between a first a position and a second position.

For the detailed structure of the interaction member 7 (e.g. regarding the first and second portion 17, 18, the rough surface 23 of the opening 7A, the bias against the first or second position etc.) and the effect of movement of the interaction member 7 between the first and second position it is referred to the description of FIGS. 4A and 4B.

In this embodiment, the opening 7A of the interaction member 7 comprises a thread that conforms to the thread 12A of the piston rod 12. In contrast to the previously described embodiment, in this embodiment the guide member 14 plays an important role. In the first position the interaction member 7 has a predetermined or first distance to the guide member 14. In particular, in the first position the interaction member 7 is not in direct mechanical contact with the guide member 14. The piston rod 12 can rotate through the opening 7A of the interaction member 7 and of the guide member 14. In the first position, the interaction member 7 may engage the piston rod 12. Alternatively, as described above, the interaction member 7 may not engage the piston rod 12 when the interaction member 7 is arranged in the first position. The contact area between the piston rod 12 and the interaction member 7 may be smaller when the interaction member 7 is in the first position than compared to the contact area when the interaction member 7 is in the second position.

For being moved from the first position into the second position, the interaction member 7 can be rotated about an angle towards the guide member 14. The interaction member 7 is rotated by moving the second portion 18. In the second position the interaction member 7 engages with the piston rod 12 such that friction is generated. In the second position, the interaction member 7 has a second distance to the guide member 14. The second distance is smaller than the predetermined or first distance. In the second position the interaction member 7 is preferably in direct mechanical contact with the guide member 14. In particular, the interaction member 7 may abut the guide member 14. In the second position the interaction member 7 works like a counter nut or jam nut.

When the interaction member 7 is rotated against the guide member 14 it is twisted against the guide member 14 and/or the piston rod 12. In the second position friction is generated due to the twisted tension. The friction reduces the speed of rotation of the piston rod 12 as described above. The amount of friction depends on the amount of rotating the interaction member 7 against the guide member 14, i.e. the angle of rotation. Hence, determining the angle of rotation allows controlling the movement of the piston rod 12. Varying the angle causes varying the friction and, therefore, allows controlling the speed of movement of the piston rod 12 as described in connection with the FIGS. 4A and 4B. Thus, when the interaction member 7 is arranged in the second position, the injection speed of the piston rod 12 is reduced as compared to the injection speed when the interaction member 7 is arranged in the first position. In this embodiment, the operation of the interaction member 7 is not independent from the function of the guide member 14. Thus, in this embodiment, a guide member 14 is needed, e.g. for achieving a rotational delivery movement of the piston rod 12.

Again, the dose setting operation is not influenced by the operation of the interaction member 7 as the piston rod 12 and the guide member 14 are not moveable during the dose setting operation.

Other implementations are within the scope of the following claims. Elements of different implementations may be combined to form implementations not specifically described herein.

### REFERENCE NUMERALS

1 Drug delivery device

2 Cartridge holder

3 Cartridge

4 Engagement means

5 Housing

5A Opening

6A Activation member

6 Dose setting member

7 Interaction member

7A Opening

8 Distal end

9 Proximal end

10 Drug

11 Bung

12 Piston rod

12A Thread

13 Bearing member

14 Guide member

15 Drive shaft

16 Energy storing member

17 Second portion

18 First portion

19 Rotation member

20 Drive member

21 Axis

22 Axis

23 Surface

24 Locking member

25 Surface normal

1. An assembly for a drug delivery device (1) comprising
a piston rod (12) adapted and arranged to be displaceable in a delivery direction for dispensing a dose of a drug (10) from the device (1),
an energy storing member (16) which is adapted and arranged to store energy and to move the piston rod (12) in the delivery direction,
an interaction member (7) adapted and arranged to mechanically cooperate with the piston rod (12), wherein the interaction member (7) is displaceable between a first position and a second position with respect to the piston rod (12), wherein the interaction member (7) is configured to be moved from the first position towards the second position to increase a friction onto the piston rod (12) and, wherein the interaction member (7) is configured to be moved from the second position back into the first position to decrease the friction onto the piston rod (12).

2. The assembly according to claim 1,
wherein, when the interaction member (7) is positioned in the first position, the piston rod (12) is displaceable in the delivery direction at a first speed and, when the interaction member (7) is moved towards the second position, the interation member (7) generates a force onto the piston rod (12), the piston rod (12) being displaceable in the delivery direction at a second speed, wherein the second speed is smaller than the first speed.

3. The assembly according to any of the previous claims,
wherein the interaction member (7) comprises a first portion (18) and a second portion (17), wherein the first portion (18) is arranged around the piston rod (12) and, wherein the second portion (17) is configured to be operated by the user.

4. The assembly according to claim 3,
further comprising a housing (5), wherein the housing (5) comprises an opening (5A) and, wherein the second portion (17) is adapted and arranged to extend through the opening (5A) of the housing (5).

5. The assembly according to claim 3 or claim 4,
wherein the second portion (17) is biased towards the first position or towards the second position.

6. The assembly according to any of claims 3 to 5,
wherein the piston rod (12) is in direct mechanical contact with the first portion (18) at least when the interation member (7) is arranged in the second position.

7. The assembly according to any of the previous claims,
wherein, for moving the interaction member from the first position into the second position, the interaction member (7) is tiltable about an axis (22) which is perpendicular to an axis (21) along which the piston rod (12) is moved during a dose delivery operation.

8. The assembly according to any of the previous claims,
wherein the interaction member (7) comprises an opening (7A), wherein, the piston rod (12) is guided through the opening (7A), and wherein, when the interaction member (7) is arranged in the first position, a surface normal (25) of the opening (7A) is parallel to the axis (22) which is perpendicular to the axis (21) along which the piston rod (12) is moved during a dose delivery operation.

9. The assembly according to claim 8,
wherein, when the interaction member (7) is arranged in the second position, the surface normal (25) of the opening (7A) is arranged at an angle (α) with respect to the axis (22) which is perpendicular to the axis (21) along which the piston rod (12) is moved during a dose delivery operation.

10. The assembly according to claim 9,
wherein an amount of friction onto the piston rod (12) depends on the size of the angle (a) between the surface normal (25) of the opening (7A) and the axis (22) perpendicular to the axis (21) along which the piston rod (12) is moved during a dose delivery operation.

11. The assembly according to any of claims 1 to 6,
further comprising a guide member (14), wherein the guide member (14) is adapted and arranged to mechanically cooperate with the piston rod (12) to guide movement of the piston rod in the delivery direction, wherein, when the interaction member (7) is positioned in the first position, it has a predetermined first distance with respect to the guide member (14).

12. The assembly according to claim 11,
wherein, when the interation member (7) is moved from the first position into the second position, the interaction member (7) is rotated by an angle towards the guide member (14) along the axis (21) along which the piston rod (12) is moved during a dose delivery operation such that the interaction member (7) is arranged at a second distance with respect to the guide member (14) when the interaction member (7) is positioned in the second position.

13. The assembly according to claim 11 or claim 12,
wherein, when the interaction member (7) is arranged in the second position, the interaction member (7) abuts the guide member (14) to increase the friction.

14. The assembly according to claim 12 or claim 13,
wherein an amount of friction onto the piston rod (12) depends on the angle by which the interaction member (7) is rotated towards the guide member (14).

15. A drug delivery device comprising the assembly according to any of the previous claims,
wherein the drug delivery device (1) further comprises a cartridge (3) for holding a plurality of doses of the drug (10) and an activation member (6A) adapted and arranged to be operated by a user for triggering a dose delivery operation of the assembly, wherein the energy storing member (16) is adapted and arranged to move the piston rod (12) in the delivery direction when the activation member (16) is operated by the user.
